(19) United States
(12) Patent Application Publication
(10) Pub. No.: US 2014/0276126 A1
(43) Pub. Date: Sep. 18, 2014

(54) METHOD AND APPARATUS FOR PROVIDING INTEGRATED MEDICAL SERVICES

(71) Applicant: GestInTime, Inc., Palo Alto, CA (US)

(72) Inventors: Suresh SUBRAMANIAM, Palo Alto, CA (US); Krishna GARIMELLA, San Jose, CA (US)

(73) Assignee: GestInTime, Inc., Palo Alto, CA (US)

(21) Appl. No.: 13/842,191

(22) Filed: Mar. 15, 2013

(51) Int. Cl. A61B 5/00; A61B 5/0205

(52) U.S. Cl. CPC A61B 5/7275; A61B 5/0205
USPC 600/483

(57) ABSTRACT

An improved method and apparatus for providing integrated medical services is disclosed.

### TECHNICAL FIELD

An improved method and apparatus for providing integrated medical services is disclosed.

### BACKGROUND OF THE INVENTION

The collection and analysis of medical data in the prior art is relatively localized. Patients typically visit their physicians, and the physicians collect and record data (such as blood pressure) at the physician's office. Patients are able to collect certain data at home, such as by using blood pressure devices that can be purchased at a pharmacy, but the prior art does not include any satisfactory mechanism for integrating the collection of medical data at the home and the physician's office. In addition, there is no automated way to share medical data collected at the home with your physician.

This problem is particularly acute during pregnancy. Data collection is prevalent and important during pregnancy. Physicians routinely collect data regarding blood pressure, weight, blood sugar, pulse, and contractions, and some women will collect such data at home. However, there is no integration between the home and physician's office. For women with “at risk” pregnancies, a woman could collect data from a medical device at home without realizing that the data indicates an alarming measurement or trend, and that data may never get communicated to a physician.

What is needed is an integrated approach to medical data collection and analysis and an improved medium by which patients and physicians can communicate.

### SUMMARY OF THE INVENTION

The aforementioned problem and needs are addressed through an embodiment for collecting patient data from the home or other site using a medical device, transferring that data to a computing device, uploading the data into a server, processing the data, generating alerts to a physician if necessary, and providing portals by which the patient and physician can communicate and exchange data and information.

### BRIEF DESCRIPTION OF THE DRAWINGS

FIG. 1 depicts an embodiment of a system for providing integrated medical services.

FIG. 2 depicts software components of a server for providing integrated medical services.

FIG. 3 depicts hardware components of an embodiment of a server.

FIG. 4 depicts an exemplary patient portal login page.

FIG. 5A depicts an exemplary patient portal welcome page.

FIG. 5B depicts an exemplary patient portal welcome page.

FIG. 6 depicts an exemplary patient portal messages page.

FIG. 7 depicts an exemplary patient portal learning page.

FIG. 8 depicts an exemplary patient portal account settings page.

FIG. 9 depicts an exemplary patient portal journal page.

FIG. 10 depicts an exemplary physician portal login page.

FIG. 11 depicts an exemplary physician portal welcome page.

FIG. 12 depicts an exemplary physician portal selected patient page.

FIG. 13 depicts an embodiment of a computing device with various display options.

FIG. 14 depicts an embodiment of display eyewear.

### DETAILED DESCRIPTION OF THE PREFERRED EMBODIMENTS

An embodiment will now be described with reference to FIG. 1. Medical device 10 collects data from a patient in the home, physician's office, or any other site. Medical device 10 can be a device to measure weight, heart rate, blood pressure, blood sugar levels, contractions, fetal heart rate, or any other device to take a measurement relevant to the health of the patient. The data is transmitted to computing device 20 over known interfaces, such as a USB connector, Bluetooth, or Wifi (e.g., 802.11).

Computing device 20 can be a desktop, notebook, server, mobile phone, tablet, game console, or any other type of device with a processor, memory, and network interface. Computing device 20 communicates with server 30 over a network 25. Network 25 optionally can be the Internet and can be hardwired, wireless, or some combination of the two. In this embodiment, computing device 20 is operated by a patient.

Server 30 is coupled to data store 50. Server 30 is also coupled to computing device 40 over network 25. Computing device 40 can be a desktop, notebook, server, mobile phone, tablet, game console, or any other type of device with a processor, memory, and network interface. In this embodiment, computing device 40 is operated by a physician.

Server 30 is coupled to computing device 60 over network 25. Computing device 60 can be a desktop, notebook, server, mobile phone, tablet, game console, or any other type of device with a processor, memory, and network interface. Computing device 60 is coupled to data store 70. In this embodiment, computing device 60 is operated by an electronic medical records (EMR) company, such as a health insurance company or billing agency.

Data store 50 and data store 70 optionally can each be a relational database for storing data records, such as a MySQL database.

With reference to FIG. 2, various software components of server 30 are depicted. Server 30 comprises risk classification engine 100, recommendation engine 110, notification engine 120, messaging engine 130, web server 140, application interface 150, and integration gateway 160.

Risk classification engine 100 comprises lines of code executed by processor 200 of server 30. Risk classification engine 100 analyzes the data collected from a patient using medical device 10, compares it against known criteria, such as those available from the American Congress of Obstetricians and Gynecologists (ACOG), and characterizes the patient in her current state with risk ratings, such as “low risk,” “medium risk,” or “high risk.” For example, a patient with an extremely high blood sugar reading could be categorized as “high risk.” The risk ratings are stored in data store 50 and are associated with the patient's profile.

Recommendation engine 100 comprises lines of code executed by processor 200 of server 30. Recommendation engine 100 generates a recommendation based on the data collected from the patient and existing data in data store 50 and makes a recommendation for a physician. For example, for a “high risk” patient, the recommendation could be “Call patient immediately,” or “Instruct patient to engage in 30 minutes of exercise to lower blood sugar level.” The recommendation is stored in data store 50 and also made known to notification engine 120 and/or messaging engine 130.

Notification engine 120 comprises lines of code executed by processor 200 of server 30. Notification engine 120 provides notifications or alerts to patients, physicians, or other persons using email, SMS or MMS messages, intra-system messages, phone calls, or on-screen alerts. For example, if recommendation engine 100 generated a recommendation of “Call patient immediately,” notification engine 120 would send that message to the physician associated with the patient in data store 50.

Messaging engine 130 comprises lines of code executed by processor 200 of server 30. It permits messages to be sent among patients, physicians, and server 30.

Web server 140 comprises lines of code executed by processor 200 of server 30. Web server 140 generates a web-based portal for patients and a web-based portal for physicians, as described in subsequent paragraphs.

Web server 140 is capable of generating web pages that are specifically suited for the particular computing device 20 or 40 that is being used. For example, different cascading style sheets can be used for a desktop computer and a mobile device, such that the web pages are optimized for display on the particular device. The underlying device can be identified, and the appropriate cascading style sheet selected, using well-known HTTP communication.

Application interface 150 comprises lines of code executed by processor 200 of server. Application interface 150 is capable of interfacing with applications running on computing device 20, computing device 40, or computing device 60 using Application Programming Interfaces (APIs), such as APIs known and used in conjunction with the iOS and Android operating systems, facebook, and Twitter.

Integration gateway 160 comprises lines of code executed by processor 200 of server 30. Integration gateway 160 interfaces with computing device 60 to exchange data relating to EMR. Computing device 60 optionally can be operated by a health insurance provider. Integration gateway 160 is designed to exchange data in a protocol and/or format that is expected or specified by the computing device 60 or its operator. For example, health insurance companies typically require that data be sent to them in a certain format with certain billing codes.

FIG. 3 depicts server 30 and some of its hardware components. Server 30 comprises a processor 200, memory 210, and network interface 220. Network interface 220 can comprise one or more wired or wireless interfaces, such as an Ethernet interface, WiFi (e.g., 802.11), WiMax, cell phone (e.g., 3G or 4G), Bluetooth, or other interface.

Patient Portal

The patient portal accessible and used by a patient will now be described. FIG. 4 shows a screenshot of an exemplary login page 300 for patients. Login page 300 is generated by server 30 and is sent to computing device 20 using web server 140 (if served as a web page) or application interface 150 (if served as a non-web browser application). Login page 300 comprises input device 310 to receive the patient's user name and input device 320 to receive the patient's password. Input device 310 and input device 320 can comprise, for example, HTML text boxes.

FIGS. 5A and 5B show screenshots of an exemplary “welcome page” 330 for a particular patient. This page might be displayed, for example, after a patient logs in using the login screen depicted in FIG. 4. Welcome page 300 comprises user input device 331 (to access messages), input device 332 (to access a learning page), input device 333 (to access account settings) and input device 334 (to logout of the server). Input devices 331, 332, 333, and 334 can be, for example, HTML buttons, tabs, or links.

Welcome page 330 comprises graphical timeline 335 that can show major health events for the patient. For example, graphical timeline 335, for a maternity patient, might show the number of trimesters that have elapsed, the temporal position within the current trimester, and major tests and events that have occurred (such as an ultrasound test). Welcome page 330 also contains an entry 336 displaying the patient's due date or upcoming appointment dates.

Welcome page 330 further comprises data box 337a, graph 337b, data box 338a, and graph 338b. Data boxes 337a, 338b, and 340 display important data regarding the patient's health, such as current or recent readings of important metrics, such as blood sugar level, weight, blood pressure, or heart rate. Graphs 337b and 338b display the data in graphical form. Optionally, graphs 337b and 338b can be displayed in animated form to show how the data has changed over time. Optionally, data boxes 3371, 338b, and 340 and graphs 337b and 338b can compare the patient's data against ideal or expected data for those items or against the patient's prior data (for example, from a previous pregnancy), or against data collected from other persons such as persons within the patient's social network.

Welcome page 330 further comprises data upload field 339. Data upload field 339 comprises a button, menu, link, or other input device that, when selected by a patient, will provide an interface by which the patient can upload or enter data from a medical device 10. For example, data upload field 339 can generate a new page with a text box and menu item to enable a patient to type in data and to indicate the type of data it is (e.g., 150 lbs, weight). Data upload field 339 also can enable computing device 20 to receive data directly from medical device 10.

Welcome page 330 further comprises input field 341 to receive information regarding the patient's current mood. In this embodiment, the patient can click on one of three icons to convey her mood (happy; sad; or in between). A patient can create a journal entry or access prior journal entries by selecting input field 342.

Welcome page 330 further comprises new messages field 343 to display any new or recent messages received from a physician or elsewhere.

FIG. 6 depicts exemplary patient portal messages page 350, such as may be accessed through input device 331 on welcome page 330. Patient portal messages page 350 comprises field 351 to display received messages; field 352 to display sent messages, field 353 to allow the patient to composes a new message; and field 354 to display any alerts generated by server 30.

FIG. 7 depicts exemplary patient portal learning page 360, such as may be accessed through input device 332 on welcome page 330. Patient portal learning page 360 comprises field 351 to display information, field 362 to suggest social network groups for the patient (based on recommendations from recommendation engine 110), field 363 to provide suggested reading materials (based on recommendations from recommendation engine 110), and field 364 to display other resources for the patient (such as videos).

FIG. 8 depicts exemplary patient portal account settings page 370, such as may be accessed through input device 333 on welcome page 330. Patient portal account settings page 360 comprises field 371 to allow the patient to edit his or her profile, such as by editing information concerning name, address, medical plan, demographics, etc.

FIG. 9 depicts exemplary patient portal journal page 380, such as may be accessed through input device 342 on welcome page 330. Patient portal account journal page 380 includes field 381 for creating a new journal entry, field 382 for reviewing old journal entries, field 383 for uploading data or files (such as photos), and social network interface 384 for sharing journal entries for uploaded data or files (such as a “post on facebook” button).

Physician Portal

The physician portal accessible and used by a physician will now be described. FIG. 10 shows a screenshot of an exemplary login page 400 for patients. Login page 400 is generated by server 30 and is sent to computing device 40 using web server 140 (if served as a web page) or application interface 150 (if served as a non-web browser application). Login page 400 comprises input device 410 to receive the patient's user name and input device 420 to receive the patient's password. Input device 410 and input device 420 can comprise, for example, HTML text boxes.

FIG. 11 shows a screenshot of an exemplary “welcome page” 430 for a particular physician. This page might be displayed, for example, after a physician logs in using the login screen depicted in FIG. 10. Welcome page 430 comprises user field 431 for selecting “all patients” or a particular patient. User field 341 might include a drop-down menu, pop-up menu, scroll entries, etc. Welcome page 430 further comprises text box 432 to enable a physician to search by name (which the physician types into the box). Welcome page 430 further comprises field 433 for patients, displaying key information for each patient (such as risk categorization, due date, and weight). Welcome page 430 further comprises text box 434 for displaying alerts, notifications, or anything else warranting the physician's immediate attention. For example, text box 434 can display notifications from notification engine 120 (“Call patient immediately”).

FIG. 12 depicts exemplary physician portal selected patient page 440. This page might be generated, for example, when the patient selects a particular patient on welcome page 430. Physician portal selected patient page 440 comprises field 441 to display alerts or notifications concerning that patient, field 442 to display the current status of the patient, field 443 for displaying a snapshot of the patient's clinical history, and field 444 for displaying any messages from or concerning that patient.

Display Options

FIG. 13 depicts computing device 40 and various mechanisms for a physician to view the physician portal or relevant pages therein. These mechanisms include a display 500 (such as an LCD), mobile device 510 (such as a tablet or mobile phone), and eyewear 520.

FIG. 14 depicts an example of eyewear 520. Eyewear 520 comprises lenses 522 and frame 521 (just as with normal glasses). But it also includes display unit 530 and processing and transmission unit 540 (embedded within the frame 521).

An example of eyewear 520 was recently announced by Google as the “Google Glass” product. Eyewear 520, such as the Google Glass, comprises a display unit 530 that displays data that you could otherwise display on an LCD or other display. For example, all of the pages described previously for the physician portal could be displayed on display unit 530.

The possible uses of eyewear 520 by physicians are numerous. For example, a physician could: (a) view pages from the physician portal on display unit 530 during a patient examination, during a remote consultation, or during a collaborative session with a fellow physician (e.g., two physicians viewing the same x-ray); (b) look at the patient in the physician's office while the display unit 530 displays patient medical data such as blood pressure, etc.; (c) apply physical pressure to the patient and get instant visual feed-back on soreness, pain points, soft-tissue, broken bones etc. The physician's view can be augmented with enhanced clinical data, such as heart rate; (d) look at a patient's hospital ID band (which gets scanned) and then view the patient's information on display unit 530; (e) Look at the patient and then have server 30 perform image/facial recognition to identify the patient and access and display his or her information in display unit 530; and (f) examine patient, and can get assisted by visual feedback critical information such as simulated images of the patient's internal organs. For example, the physician would be able to identify the location of the patient's spleen and then feel the spleen, because he or she could see the exact location of the spleen as a visual overlay of the patient's internal organ structure on the patient.

References to the present invention herein are not intended to limit the scope of any claim or claim term, but instead merely make reference to one or more features that may be covered by one or more of the claims. Materials, processes and numerical examples described above are exemplary only, and should not be deemed to limit the claims. It should be noted that, as used herein, the terms “over” and “on” both inclusively include “directly on” (no intermediate materials, elements or space disposed there between) and “indirectly on” (intermediate materials, elements or space disposed there between). Likewise, the term “adjacent” includes “directly adjacent” (no intermediate materials, elements or space disposed there between) and “indirectly adjacent” (intermediate materials, elements or space disposed there between). For example, forming an element “over a substrate” can include forming the element directly on the substrate with no intermediate materials/elements there between, as well as forming the element indirectly on the substrate with one or more intermediate materials/elements there between.

What is claimed is:

1. A server for providing interactive medical services, comprising:
a web server for generating a first web portal and a second web portal;
a risk classification engine for generating risk classification information for a patient based on medical device data received through the first web portal; and
a notification engine for providing the risk classification information through the second web portal.

2. The server of claim 1, wherein the first web portal provides medical information for the patient.

3. The server of claim 2, wherein the medical information includes graphical data concerning one or more of heart rate, blood pressure, weight, and blood sugar level.

4. The server of claim 3, wherein the graphical data provides information concerning expected values for one or more of heart rate, blood pressure, weight, and blood sugar level.

5. The server of claim 1, wherein the risk classification information comprises one of at least three risk classifications.

6. The server of claim 1 further comprising an integration gateway for communicating with an electronic medical records server.

7. The server of claim 1 further comprising an application interface for communicating with an application on a computing device using an application programming interface (API).

8. A system for providing interactive medical services, comprising:
a server;
a first computing device for displaying a first portal and for communicating with the server; and
a second computing device for displaying a second portal and for communicating with the server;
wherein the server comprises a risk classification engine for generating risk classification information for a patient based on medical device data received through the first portal and a notification engine for providing the risk classification information through the second portal.

9. The system of claim 8, wherein the first portal provides medical information for the patient.

10. The system of claim 9, wherein the medical information includes graphical data concerning one or more of heart rate, blood pressure, weight, and blood sugar level.

11. The system of claim 10, wherein the graphical data provides information concerning expected values for one or more of heart rate, blood pressure, weight, and blood sugar level.

12. The system of claim 8, wherein the risk classification information comprises one of at least three risk classifications.

13. The system of claim 8, wherein the server further comprises an integration gateway for communicating with an electronic medical records server.

14. The system of claim 8, wherein the first portal is configured by an application interface running on the server through an application programming interface (API).

15. The system of claim 8, wherein the second computing device is coupled to an eyewear device comprising a display.

16. The system of claim 15, wherein the display is capable of displaying the second portal.

17. A method of providing medical services, comprising:
generating, by a web server running within a server, a first web portal and a second web portal;
generating, by a recommendation engine running within the server, a recommendation based on the medical device data received through the first web portal; and
providing, by a notification engine running within the server, the recommendation through the second web portal.

18. The method of claim 17, further comprising displaying in the first web portal graphical data concerning one or more of heart rate, blood pressure, weight, and blood sugar level.

19. The method of claim 18, wherein the graphical data provides information concerning expected values for one or more of heart rate, blood pressure, weight, and blood sugar level.

20. The method of claim 17, further comprising generating, by a risk classification engine, risk classification information based on the medical device data received through the first web portal.
